SEQUENCE LISTING (24 sequences)

SEQ ID NO: 1 - DNA, 1901 bp, Homo Sapiens
gggcaggaagacggcgctgcccggaggagcggggcgggcgggcgcgcgggggagcgggcggcgggcgggagccaggcccgggcgggggcgggggcggcggggccagaagaggcggcgggccgcgctccggccggtctgcggcgttggccttggctttggctttggcggcggcggtggagaagatgctgcagtccctggccggcagctcgtgcgtgcgcctggtggagcggcaccgctcggcctggtgcttcggcttcctggtgctgggctacttgctctacctggtcttcggcgcagtggtcttctcctcggtggagctgccctatgaggacctgctgcgccaggagctgcgcaagctgaagcgacgcttcttggaggagcacgagtgcctgtctgagcagcagctggagcagttcctgggccgggtgctggaggccagcaactacggcgtgtcggtgctcagcaacgcctcgggcaactggaactgggacttcacctccgcgctcttcttcgccagcaccgtgctctccaccacaggttatggccacaccgtgcccttgtcagatggaggtaaggccttctgcatcatctactccgtcattggcattcccttcaccctcctgttcctgacggctgtggtccagcgcatcaccgtgcacgtcacccgcaggccggtcctctacttccacatccgctggggcttctccaagcaggtggtggccatcgtccatgccgtgctccttgggtttgtcactgtgtcctgcttcttcttcatcccggccgctgtcttctcagtcctggaggatgactggaacttcctggaatccttttatttttgttttatttccctgagcaccattggcctgggggattatgtgcctggggaaggctacaatcaaaaattcagagagctctataagattgggatcacgtgttacctgctacttggccttattgccatgttggtagttctggaaaccttctgtgaactccatgagctgaaaaaattcagaaaaatgttctatgtgaagaaggacaaggacgaggatcaggtgcacatcatagagcatgaccaactgtccttctcctcgatcacagaccaggcagctggcatgaaagaggaccagaagcaaaatgagccttttgtggccacccagtcatctgcctgcgtggatggccctgcaaaccattgagcgtaggatttgttgcattatgctagagcaccagggtcagggtgcaaggaagaggcttaagtatgttcatttttatcagaatgcaaaagcgaaaattatgtcactttaagaaatagctactgtttgcaatgtcttattaaaaaacaacaaaaaaagacacatggaacaaagaagctgtgaccccagcaggatgtctaatatgtgaggaaatgagatgtccacctaaaattcatatgtgacaaaattatctcgaccttacataggaggagaatacttgaagcagtatgctgctgtggttagaagcagattttatacttttaactggaaactttggggtttgcatttagatcatttagctgatggctaaatagcaaaatttatatttagaagcaaaaaaaaaaagcatagagatgtgttttataaataggtttatgtgtactggtttgcatgtacccacccaaaatgattatttttggagaatctaagtcaaactcactatttataatgcataggtaaccattaactatgtacatataaagtataaatatgtttatattctgtacatatggtttaggtcaccagatcctagtgtagttctgaaactaagactatagatattttgtttcttttgatttctctttatactaaagaatccagagttgctacaataaaataaggggaataataaacttgagagtgaataaccat

SEQ ID NO: 2 - DNA, 22 bp, artificial sequence
  primer 1
tgaagaaggacaaggacgagga

SEQ ID NO: 3 - DNA, 20 bp, Artificial sequence
  primer 2
gcctggtctgtgatcgagga

SEQ ID NO: 4 - DNA, 27 bp, artificial sequence
  probe
caggtgcacatcatagagcatgaccaa

SEQ ID NO: 5 - DNA, 2590 bp, Homo Sapiens
tgccctgcgcggagagcggcgagcgcagccatgccccaggccgcctccggggcagcagcagcggcggccggggccgatgcgcgggccgggggcgccggggggccggcggcggcccgggcgggacgatgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagcctgtccaccttccgcggcctcatgaagcgcaggagctccgtgtgactgccccgagggacctggagcacctgggggcgcgggcgggggacccctgctgggaggccaggagactgcccctgctgccttctgcccagtgggaccccgcacaacatccctcaccactctcccccagcacccccatctccgactgtgcctgcttgcaccagccggcaggaggccgggctctgaggacccctggggcccccatcggagccctgcaaattccgagaaatgtgaaacttggtggggtcagggaggaaaggcagaagctgggagcctcccttccctttgaaaatctaagaagctcccagtcctcagagaccctgctggtaccacaccccaccttcggaggggacttcatgttccgtgtacgtttgcatctctatttatacctctgtcctgctaggtctcccaccttcccttggttccaaaagccagggtgtctatgtccaagtcacccctactcagccccactccccttcctcatccccagctgtgtctcccaacctcccttcgtgttgttttgcatggctttgcagttatggagaaagtggaaacccagcagtccctaaagctggtccccagaaagcaggacagaaagaaggagggacaggcaggcagcaggaggggcgagctgggaggcaggaggcagcggcctgtcagtctgcagaatggtcgcactggaggttcaagctaactggcctccagccacattctcatagcaggtaggacttcagccttccagacactgcccttagaatctggaacagaagacttcagactcaccataattgctgataattacccactcttaaatttgtcgagtgatttttagcctctgaaaactctatgctggccactgattcctttgagtctcacaaaaccctacttaggtcatcagggcaggagttctcactcccattttacagatgagaatactgaggcctggacaggtgaagtgaccagagagcaaaaggcaaaggggtgggggctgggtgcagtggctcacacctgtattcccaacacttttggaggctgaggttggaggattgcttgagcccaggaattcgagaccagcctaggtgacatagtgagaccccatctctacaaaaaataaaaaattaaccaggtgtggtggcacgtgcctgggagtcccagcgacttgggaggctgaggtgggaggattgtttgagcctgggaggtcgaggctgtagtgagccctgattgcaccactgtactccagcctgggtgacagggcaagaccctgtctcaaaaaaaaaaaaaaa

SEQ ID NO: 6 - DNA, 19 bp, artificial sequence
  primer 1
acgtctacgcggaggtgct

SEQ ID NO: 7 - DNA, 18 bp, artificial sequence
  primer 2
tctcgcggctcttgtacc

SEQ ID NO: 8 - DNA, 26 bp, artificial sequence
  probe
cacttccagtccatgtgctcgtgcct

SEQ ID NO: 9 - DNA, 2890 bp, Homo Sapiens
ctccgtcccaggggagaaggagaggcgtctgcagggggcagagaccgcagctacctgccgggtgcgccccccacccaggagcgctcgcttcgccccctttcctcccccgcccccacctccttattggtgctagtttgcagcgcccagctcctgcgccttcgcttcgcgtttgaatctggctcgccccttcgtattatgtctgcactccgaaggaaatttggggacgattatcaggtagtgaccacatcgtccagcggctcgggcttgcagccccaggggccaggccaggaccctcagcagcagcttgtgcccaagaagaagcggcagcggttcgtggacaagaacggccggtgcaatgtacagcacggcaacctgggcagcgagacaagccgctacctctcggacctcttcaccacgctggtggacctcaagtggcgctggaacctcttcatcttcattctcacctacaccgtggcctggcttttcatggcgtccatgtggtgggtgatcgcctacactcggggcgacctgaacaaagcccacgtcggtaactacacgccttgcgtggccaatgtctataacttcccttctgccttcctcttcttcatcgagacggaggccaccatcggctatggctaccgatacatcacagacaagtgccccgagggcatcatcctcttcctcttccagtccatcctgggctccatcgtggacgccttcctcatcggctgcatgttcatcaagatgtcccagcccaagaagcgcgccgagaccctcatgttcagcgagcacgcggtgatctccatgagggacggaaaactcacgcttatgttccgggtgggcaacctgcgcaacagccacatggtctccgcgcagattcgctgcaagctgctcaaatctcggcagacacctgagggtgagttccttccccttgaccaacttgaactggatgtaggttttagtacaggggcagatcaactttttcttgtgtcccccctcacaatttgccacgtgatcgatgccaaaagccccttttatgacctatcccagcgaagcatgcaaactgaacagttcgagattgtcgtcatcctagaaggcattgtggaaacaactgggatgacttgtcaagctcgaacatcatatactgaagatgaagttctttggggtcatcgtttttttcctgtaatttccttagaagagggattctttaaagttgattactcccagttccatgcaacatttgaagtccccaccccaccttacagtgtgaaagagcaggaggaaatgcttctcatgtcgtcccctttaatagcaccagccataactaacagcaaagaaagacataattctgtggaatgcttagatggactagatgatattactacaaaactaccatctaagctgcagaaaattactggaagagaagactttcccaaaaaactcttgaggatgagttctacaacttcagaaaaagcctacagcttgggagacttgcccatgaaacttcaacgaataagttcagttccgggcaactcagaagaaaaactggtatctaaaaccaccaagatgttatctgatcccatgagccagtctgtggctgatttgccaccaaagcttcaaaagatggctggaggagcagctaggatggaagggaaccttccagccaaattaagaaaaatgaactctgatcgcttcacataacaaagcactcccttaggcattatttaatgtttgatttagtaatagtccaatatttggcgatgaggtaattctccctaaggaatctgaaagtatattttcctcccagttctacaagcatatttgagaacccttcctttcccaagtattgcgaatgtgcagaaagcaacagttacggagggaggacatcataaggaagttattaacgggcatgtattatcacatcaagcatgcaataatgtgcaaattttgcatttagttttatggcatgatttatatatggcatatttatattgtatattctggaaaaaaaatatatatatatatttaaaggggagatactctccctgacatttctaacatatgtattaagccaaacatgagtgaatagctttcagggcgataaaactaaatatatgtctgtgtgtgtgtgtgtatgtatacacacatatacatatatatatacacatacatacacatacatacatacatacatatatatctgataaaattgtgatgttttgttcaaagttgtagttcttgtgcatgtttactttattagagtaggaaggctactggcattaattattaataccaaatattttagccttaaatttttgtcattttaaaatctgatttaatgttttctgctgtttaaggtcttgggaggctttcaattgtattttatatgagagaatcacacaagtttgtgctatctatggccctgcaaaaatataaccattacatgtttaaattgtaaattttagagcataccagtactcagtatagcattgaacatttcttatgatttttaaaagttgctagtactggggagaaataattgttgattaatttgagaattattcctttcctagactaattaaaatctggaaatctgttttgtatatgatctaatacaaagatgagctctgaacaaacactgaatcatgttaatagacagtagccaagttatattgaatatatcagaatctgtgtgaagttacacaattaattgtccctgtttcaaactgagtaaattggaaacattttctttctttttctggaaattttgtccattttaaaaaccaatcattttaagaagacatgacaatgcaatgaaacagatgataaatatttatgcttaaaatatgtatgtctaattgagtctcttttttattctgttttcttgttt

SEQ ID NO: 10 - DNA, 20 bp, artificial sequence
  primer 1
gttccacgcaacatttgaag

SEQ ID NO: 11 - DNA, 20 bp, artificial sequence
  primer 2
gggacgacatgagaagcatt

SEQ ID NO: 12 - DNA, 24 bp, artificial sequence
  probe
cccaccccaccttacagtgtgaaa

SEQ ID NO: 13 - DNA, 2510 bp, Homo Sapiens
cggcggcagcagcccatgcctccggtgcaacagctgcgcctcctccggtgccccggcggcgggggcgggagataacctgtccctgctgctccgcacctcctcgcccggcggcgccttccggacccgcacctcctcgccgctgtcgggctcgtcctgctgctgctgctgctgctcgtcgcgccggggcagccagctcaatgtgagcgagctgacgccgtccagccatgccagtgcgctccggcagcagtacgcgcagcagtccgcgcagcagtcggcgtccgcctcccagtaccaccagtgccacagcctgcagcccgccgccagccccacgggcagcctcggcagtctgggctccgcgcccccgctctcgcaccaccaccaccacccgcacccggcgcaccaccagcaccaccagccccaggcgcgccgcgagagcaaccccttcaccgaaatagccatgagcagctgcaggtacaacgggggcgtcatgcggccgctcagcaacttgagcgcgtcccgccggaacctccacgagatggactcagaggcgcagcccctgcagccccccgcgtctgtcggaggaggtggcggcgcgtcctccccgtctgcagacgctgccgccgccgccgctgtttcgtcctcagcccccgagatcgtggtgtctaagcccgagcacaacaactccaacaacctggcgctctatggaaccggcggcggaggcagcactggaggaggcggcggcggtggagggagcgggcacggcagcagcagtggcaccaagtccagcaaaaagaaaaaccagaacatcggctacaagctgggccaccggcgcgccctgttcgaaaagcgcaagcggctcagcgactacgcgctcatcttcggcatgttcggcatcgtggtcatggtcatcgagaccgagctgtcgtggggcgcctacgacaaggcgtcgctgtattccttagctctgaaatgccttatcagtctctccacgatcatcctgctcggtctgatcatcgtgtaccacgccagggaaatacagttgttcatggtggacaatggagcagatgactggagaatagccatgacttatgagcgtattttcttcatctgcttggaaatactggtgtgtgctattcatcccatacctgggaattatacattcacatggacggcccggcttgccttctcctatgccccatccacaaccaccgctgatgtggatattattttatctataccaatgttcttaagactctatctgattgccagagtcatgcttttacatagcaaacttttcactgatgcctcctctagaagcattggagcacttaataagataaacttcaatacacgttttgttatgaagactttaatgactatatgcccaggaactgtactcttggtttttagtatctcattatggataattgccgcatggactgtccgagcttgtgaaaggtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctagaagagaataagttaaccacaaaataagactttttgccatcatatggtcaatattttagcttttattgtaaagcccctatggttctaatcagcgttatccgggttctgatgtcagaatcctgggaacctgaacactaagttttaggccaaaatgagtgaaaactctttttttttctttcagatgcacagggaatgcacctattattgctatatagattgttcctcctgtaatttcactaactttttattcatgcacttcaaacaaactttactactacattatatgatatataataaaaaaagttaatttcgga

SEQ ID NO: 14 - DNA, 19 bp, artificial sequence
  primer 1
tgcacagccctggtggtag

SEQ ID NO: 15 - DNA, 21 bp, Artificial Sequence
  primer 2
tccatcatgaaattgtgcacg

SEQ ID NO: 16 - DNA, 31 bp, artificial sequence
  probe
tggcaaggaagctagaacttaccaaagcaga

SEQ ID NO: 17 - DNA, 2865 bp, Homo Sapiens
ttttcggctgcttggtaacgggctgccagaagagagagaggcagagagcagggcagcggcttcttgacgtcagggccaagcgaggggatcgcgccagcaaccccagctctccccagagaggggccggccgaccgctggagcggagcctgacgccaggcgcccgcggagcgtgagtagggggcgcgggagccggtcagctggggcgcagcatgccctctgctcccgcgccatggagatcgccctggtgcccctggagaacggcggtgccatgaccgtcagaggaggcgatgaggcccgggcaggctgcggccaggccacagggggagagctccagtgtcccccgacggctgggctcagcgatgggcccaaggagccggcgccaaaggggcgcggcgcgcagagagacgcggactcgggagtgcggcccttgcctccgctgccggacccgggagtgcggcccttgcctccgctgccagaggagctgccacggcctcgacggccgcctcccgaggacgaggaggaagaaggcgatcccggcctgggcacggtggaggaccaggctctgggcacggcgtccctgcaccaccagcgcgtccacatcaacatctccgggctgcgctttgagacgcagctgggcaccctggcgcagttccccaacacactcctgggggaccccgccaagcgcctgcgctacttcgaccccctgaggaacgagtacttcttcgaccgcaaccggcccagcttcgacggtatcctctactactaccagtccgggggccgcctgcggaggccggtcaacgtctccctggacgtgttcgcggacgagatacgcttctaccagctgggggacgaggccatggagcgcttccgcgaggatgagggcttcattaaagaagaggagaagcccctgccccgcaacgagttccagcgccaggtgtggcttatcttcgagtatccggagagctctgggtccgcgcgggccatcgccatcgtctcggtcttggttatcctcatctccatcatcaccttctgcttggagaccctgcctgagttcagggatgaacgtgagctgctccgccaccctccggcgccccaccagcctcccgcgcccgcccctggggccaacggcagcggggtcatggccccgccctctggccctacggtggcaccgctcctgcccaggaccctggccgaccccttcttcatcgtggagaccacgtgcgtcatctggttcaccttcgagctgctcgtgcgcttcttcgcctgccccagcaaggcagggttctcccggaacatcatgaacatcatcgatgtggtggccatcttcccctacttcatcaccctgggcaccgaactggcagagcagcagccagggggtggaggaggcggccagaatgggcagcaggccatgtccctggccatcctccgagtcatccgcctggtccgggtgttccgcatcttcaagctctcccgccactccaaggggctgcagatcctgggcaagaccttgcaggcctccatgagggagctggggctgctcatcttcttcctcttcatcggggtcatcctcttctccagtgccgtctacttcgcagaggctgacaaccagggaacccatttctctagcatccctgacgccttctggtgggcagtggtcaccatgaccactgtgggctacggggacatgaggcccatcactgttgggggcaagatcgtgggctcgctgtgtgccatcgccggggtcctcaccattgccctgcctgtgcccgtcatcgtctccaacttcaactacttctaccaccgggaaacggatcacgaggagccggcagtccttaaggaagagcagggcactcagagccaggggccggggctggacagaggagtccagcggaaggtcagcgggagcaggggatccttctgcaaggctggggggaccctggagaatgcagacagtgcccgaaggggcagctgccccctagagaagtgtaacgtcaaggccaagagcaacgtggacttgcggaggtccctttatgccctctgcctggacaccagccgggaaacagatttgtgaaaggagattcaggcagactggtggcagtggagtagggaatgggaggcttgctgaacatggatatctacattataccgcagagtatttgaagtcacactgtaacctcagtctacccctctcctttcactcctttcctccctccctcgatccccccattttctctattctttccatgacacccaagggtcgcctatttttaaaaagtaccacattccatgacgcaggagctgtggaaatggtgagcgctgtgagatggatgtatttgtagccagtctcctatacccagcagagggataacccaaacaaaaatgactctaaatagcccagatcccaagagattatgtaactcctccatccatgtgttccaaatttgctttacatatgattgtatttgtgtataggggaaaatattatttttatgcctggtaagtggctttttgtactgtagttcagatagagatattttgggtatattttcaagatacatgttgtatttatggaagaaagagttgtcctgatgtttttctgtgttacttatattagagtcagagatcttggtatgggctgttctgtttcctgtgtctccaagcctctgtcttttctgggatgtggtattggtgctttgtgtctagggcagagtatgttcttgaagaaaggcaaatctgactttttctgtgcgccttaaacaattcttgtaactttcttcaaaaagcattttaatgatattggaggaatacttctgataatttattgtctttatttttatcccaggaaataaaaggttaccttgttga

SEQ ID NO: 18 - DNA, 23 bp, artificial sequence
  primer 1
cagggaacccatttctctagcat

SEQ ID NO: 19 - DNA, 19 bp, artificial sequence
  primer 2
tgtccccgtagcccacagt

SEQ ID NO: 20 - DNA, 23 bp, ARTIFICIAL SEQUENCE
  probe
acgccttctggtgggcagtggtc

SEQ ID NO: 21 - DNA, 1793 bp, HOMO SAPIENS
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 22 - DNA, 20 bp, ARTIFICIAL SEQUENCE
  primer 1
tccaccttccagcagatgtg

SEQ ID NO: 23 - DNA, 21 bp, ARTIFICIAL SEQUENCE
  primer2
ctagaagcatttgcggtggac

SEQ ID NO: 24 - DNA, 28 bp, ARTIFICIAL SEQUENCE
  probe
atcagcaagcaggagtatgacgagtccg

(19) United States
(12) Patent Application Publication
(10) Pub. No.: US 2006/0183665 A1
(43) Pub. Date: Aug. 17, 2006

(54) Modulators of the potassium channels twik-1, task-1 gorl1. sk2 pr pcn1, used to treat arrhythmia, coronary heat disease or hypertension

(75) Inventors: Peter Ellinghaus, Wuppertal (DE); Klaus Munter, Wilfrath (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/565,185

(22) PCT Filed: Jul. 6, 2004
(86) PCT No.: PCT/EP04/07364

§ 371 Date: Apr. 10, 2006

(30) Foreign Application Priority Data
Jul. 18, 2003 (DE) 103 32 685.5

(51) Int. Cl. A61K 38/17

(52) U.S. Cl. 514/2

(57) ABSTRACT

The invention relates to the use of modulators of the potassium channels TWIK-1, TASK-1, GIRK1 SK2 and PCN1 for producing a medicament for the treatment and/or prophylaxis of cardiac dysrhythmias (arrhythmias), coronary heart disease, and hypertension.

The invention relates to the use of potassium channel modulators for producing a medicament for the treatment and/or prophylaxis of cardiac dysrhythmias, coronary heart disease and hypertension or a combination of said disorders.

The cells of the sinoatrial node in the right atrium of the heart have the function of a physiological pacemaker because an electrical stimulation originates there at regular intervals. A change in membrane potential which is determined by the concentration of various ions on both sides of a cell membrane (Na⁺, K⁺ and Ca²⁺) is responsible for conduction. These ions cross the cell membrane through ion-selective channels which consist of a plurality of subunits and together form a pore. During a heart action (systole), the myocardial cell passes through an action potential which is composed of phases 0-3 and in which all three of the abovementioned types of ion channels are involved. The action begins with a rapid depolarization (phase 0) in which Na⁺ channels are particularly involved, followed by a transient, incomplete repolarization (phase 1) which passes into the long-lasting plateau phase (phase 2) and in which Ca²⁺ channels are particularly involved. Phase 3 represents the repolarization and is thus responsible for restoration of the resting state. The K⁺ efflux necessary for repolarization is mediated by potassium channels. Throughout the action potential, the membrane is protected from a further depolarizing stimulus, it is refractory (1).

Arrhythmias are associated either with disturbances of depolarization, of conduction or of a combination of the two. These may be caused by ischemias, inflammatory disorders of the myocardium or else toxic effects or autonomic influences. Substances and methods influencing depolarization or conduction are employed therapeutically for the treatment of arrhythmias. Substances which delay the repolarizing K⁺ current and thus prolong the action potential duration and refractory period belong to the so-called class III antiarrhythmics, of which at present amiodarone and sotalol are authorized in Germany (1).

However, neither of the substances is a selective potassium channel blocker. Thus, sotalol shows, besides blockade of various K⁺ channels (e.g. HERG), also antagonistic properties for beta-adrenergic receptors, while amiodarone blocks, besides HERG, also the L-type Ca²⁺ channel and Na⁺ channels (1), (2).

Just like the other classes of antiarrhythmics, class III potassium channel blockers also have a considerable proarrhythmic potential which is attributed to the simultaneous influence on the potassium channels in the ventricle and limits clinical use. The identification of potassium channels which are preferentially expressed in the atrium as possible targets for antiarrhythmics thus assumes particular importance, because the side effects, which may extend to fatal ventricular fibrillation, can be reduced thereby (3).

Besides potassium channel blockers such as sotalol and amiodarone, anti-arrhythmic effects have also been described for potassium channel openers, e.g. for the ATP-dependent potassium channel (4).

In the present study, Affymetrix microarray technology was used to identify genes which are expressed in the human heart differentially between left atrium and left ventricle (see FIG. 1). Verification of the differential expression of selected genes took place by real-time PCR (TaqMan). This revealed that in all 6 investigated patients the potassium channels TWIK-1 (5), TASK-1(6), GIRK1 (7), SK2 (8) and PCN1 (9) are expressed distinctly more strongly in the atrium than in the ventricle (see FIG. 3).

The present invention therefore relates the use of modulators of the aforementioned potassium channels for producing a medicament for the treatment and/or prophylaxis of the abovementioned diseases.

Potassium channel modulators within the meaning of the present disclosure are substances which prolong or shorten the duration of opening of said potassium channels.

Modulators in the context of the invention are all substances which bring about a change in the biological activity of the channels. Particularly preferred modulators are nucleic acids, including locked nucleic acids, peptide nucleic acids, and Spiegelmers, proteins, including antibodies, and low molecular weight substances, and very particularly preferred modulators are low molecular weight substances.

The invention relates to the use of modulators of the potassium channels TWIK-1, TASK-1, GIRK1, SK2 or PCN1 for producing a medicament for the treatment and/or prophylaxis of cardiac dysrhythmias (arrhythmias), coronary heart disease or hypertension. The invention further relates to the use of modulators of the potassium channels TWIK-1, TASK-1, GIRK1, SK2 or PCN1 having an IC₅₀ of <1 μm, particularly preferably of <100 nM for producing a medicament for the treatment and/or prophylaxis of cardiac dysrhythmias (arrhythmias), coronary heart disease or hypertension.

A further aspect of the invention relates to a method for screening test compounds to identify modulators of the potassium channels TWIK-1, TASK-1, GIRK1, SK2 or PCN1 which are suitable for producing a medicament for the treatment and/or prophylaxis of cardiac dysrhythmias (arrhythmias), coronary heart disease or hypertension.

The invention likewise relates to a pharmaceutical composition comprising a modulator or a plurality of modulators of the potassium channels TWIK-1, TASK-1, GIRK1, SK2 or PCN1 for the treatment and/or prophylaxis of cardiac dysrhythmias (arrhythmias), coronary heart disease or hypertension.

A further aspect of the invention is the use of modulators of the potassium channels TWIK-1, TASK-1, GIRK1, SK2 or PCN1 for controlling the activity of the corresponding potassium channels in a living creature including a human for the treatment and/or prophylaxis of cardiac dysrhythmias (arrhythmias), coronary heart disease or hypertension.

The invention also relates to modulators of the potassium channels TWIK-1, TASK-1, GIRK1, SK2 or PCN1 for the treatment and/or prophylaxis of cardiac dysrhythmias (arrhythmias), coronary heart disease or hypertension.

Also according to the invention is the use of modulators of gene products which are expressed in the human heart differentially between left atrium and left ventricle for producing a medicament for the treatment of arrhythmias, coronary heart disease, hypertension and the sequelae of atherosclerosis. Since, depending on the function of the gene product, it is perfectly possible for enhanced expression in the ventricle also to be preferred (e.g. for the endothelin A receptor), the term differential gene expression is used herein.

A further aspect of the invention is a method for screening test compounds to identify modulators of gene products which are expressed in the human heart differentially between left atrium and left ventricle and which are suitable for producing a medicament for the treatment and/or prophylaxis of cardiac dysrhythmias (arrhythmias), coronary heart disease or hypertension.

The invention likewise relates to a pharmaceutical composition comprising a modulator or a plurality of modulators of gene products which are expressed in the human heart differentially between left atrium and left ventricle for the treatment and/or prophylaxis of cardiac dysrhythmias (arrhythmias), coronary heart disease or hypertension.

The invention further relates to the use of modulators of gene products which are expressed in the human heart differentially between left atrium and left ventricle for controlling the activity of the corresponding gene products in a living creature including a human for the treatment and/or prophylaxis of cardiac dysrhythmias (arrhythmias), coronary heart disease or hypertension.

The invention also relates to modulators of gene products which are expressed in the human heart differentially between left atrium and left ventricle for the treatment and/or prophylaxis of cardiac dysrhythmias (arrhythmias), coronary heart disease or hypertension.

Substances which have a modulating effect on the activity of said channels can be identified by the assay described below (screening).

The anti-arrhythmic effect is tested in vivo by the animal experiment described below.

### DESCRIPTION OF THE FIGURES

FIG. 1: The table lists genes which were found in all 6 investigated patients consistently to be differentially expressed between atrium and Ventricle.

FIG. 2: The table lists the Genbank accession numbers of the genes verified by TaqMan PCR, and the primer/probe sequences used therefore.

FIG. 3: The relative mRNA expression of the potassium channels TWIK-1, TASK-1, GIRK1 SK2 and PCN1 in the human heart (left atrium [black] and left ventricle [white] is shown.

FIG. 4: The relative protein expression of the potassium channel TASK-1 in human hearts as average for all 6 patients is shown. (Left atrium [black] and left ventricle [white].

### EXAMPLES

### Example 1

### Identification of Genes Expressed Differentially Between Human Ventricle and Atrium

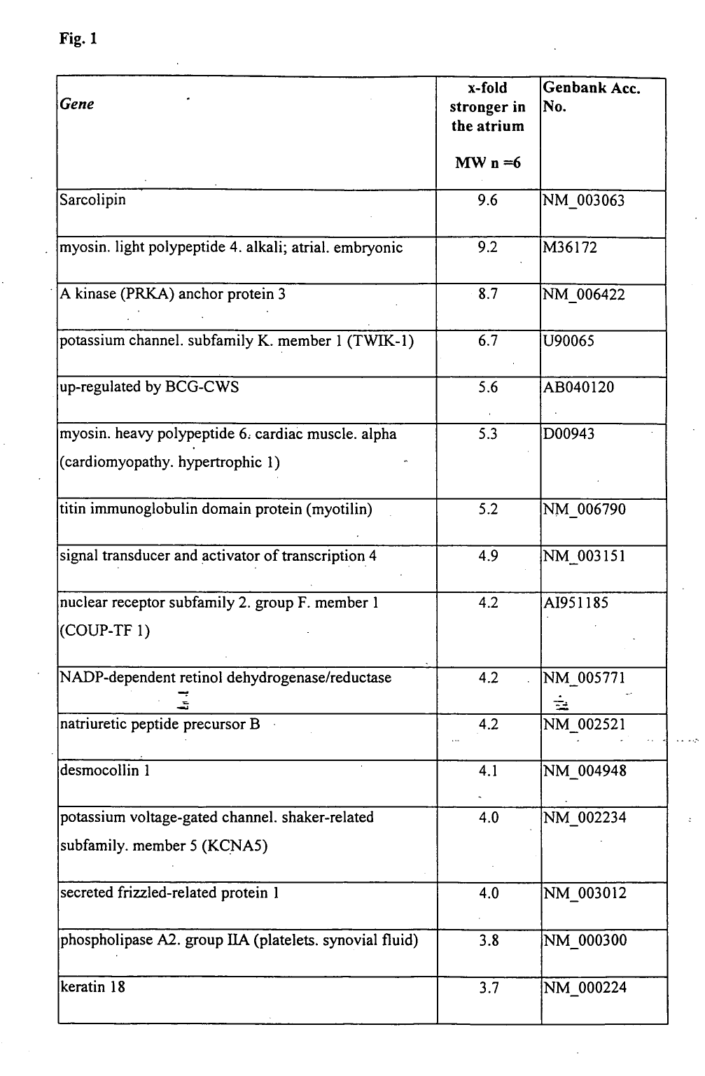

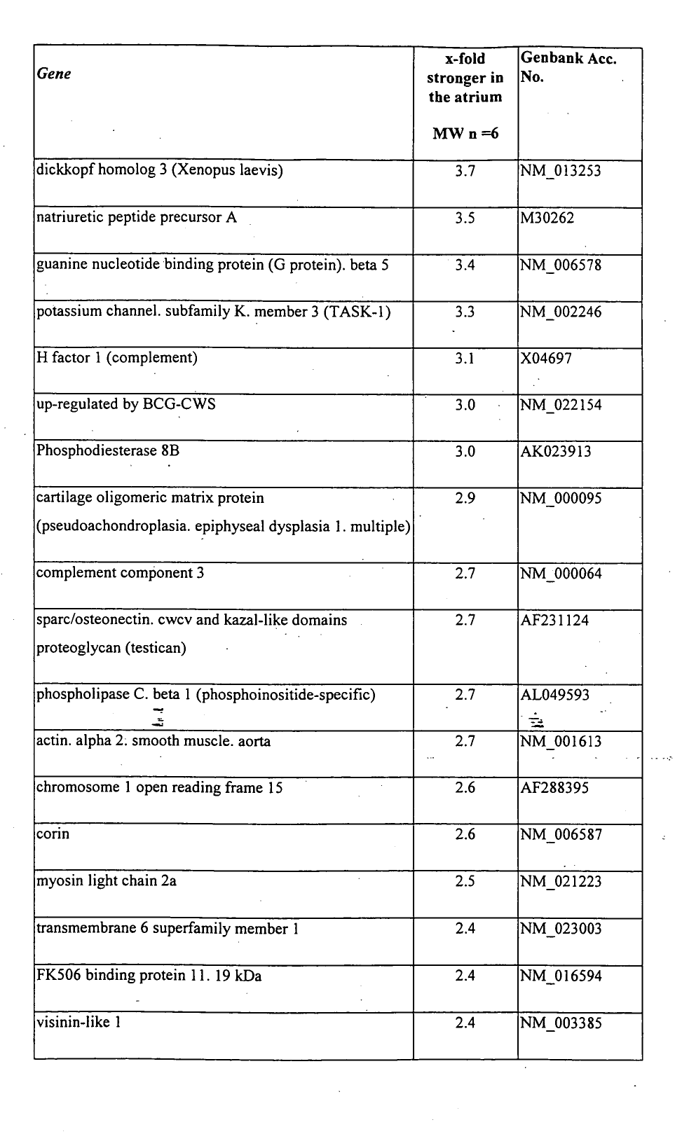

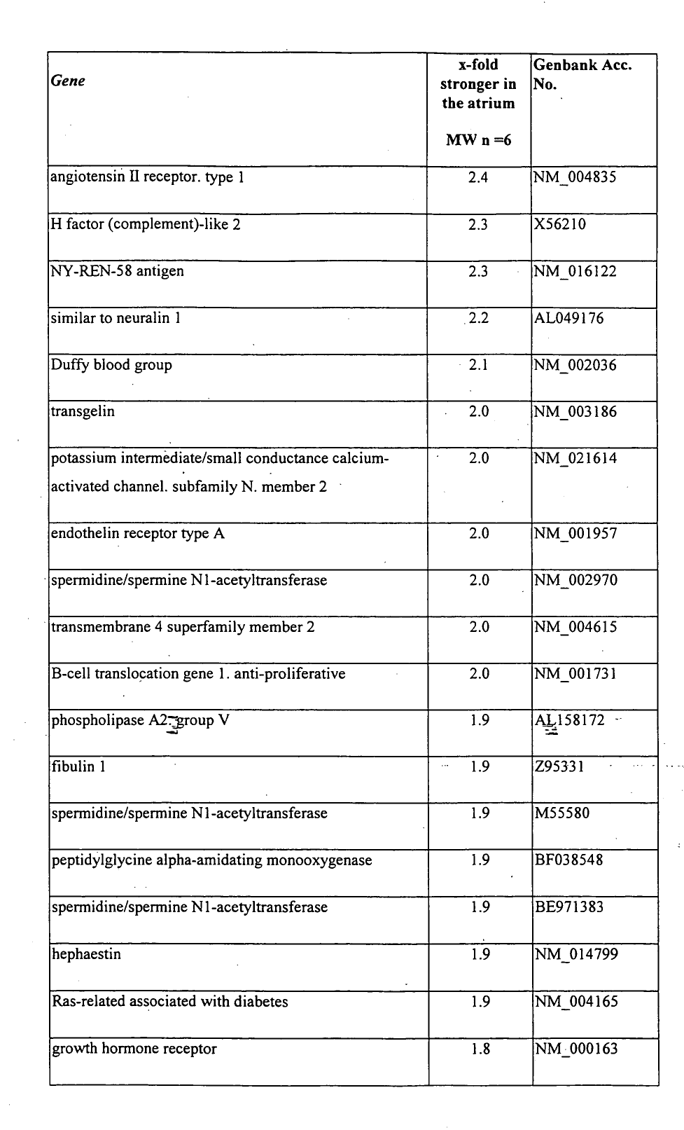

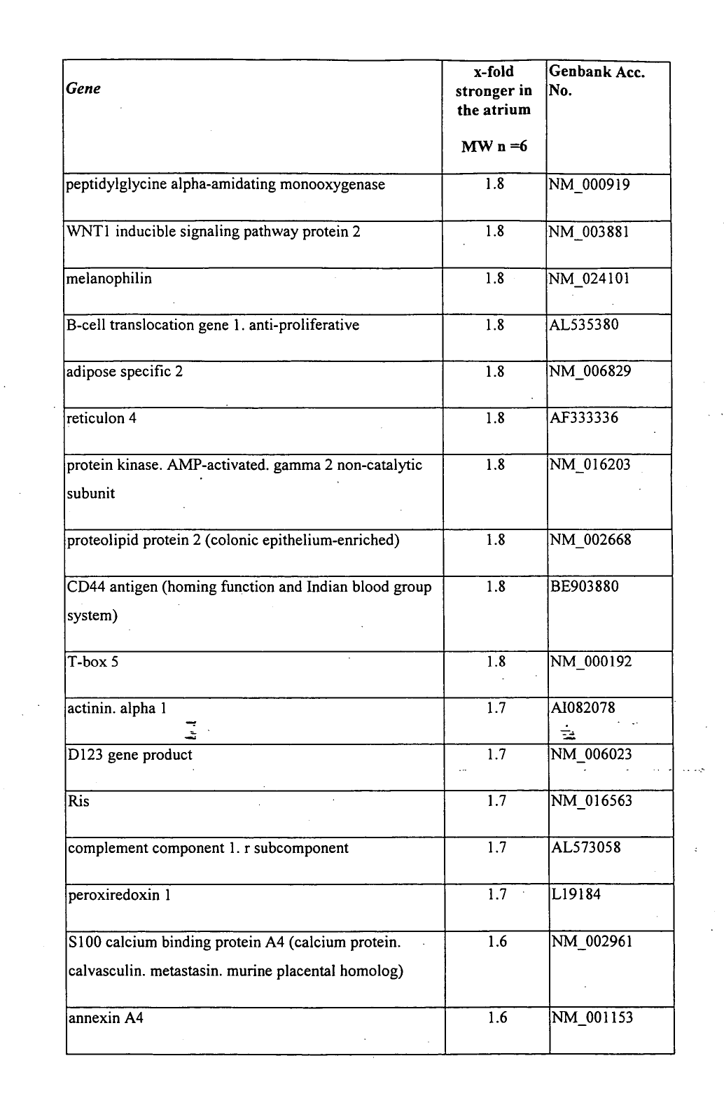

Small pieces (about 0.5 g) of the left ventricle and of the left atrium of explanted hearts were obtained with the consent of the donors from the Herzzentrum Halle (Prof. Morawietz). The total RNA was isolated therefrom after homogenization of the tissue using RNaesy columns (from Qiagen) in accordance with the instructions. Transcription of in each case 10 μg of total RNA into cDNA, subsequent linear amplification thereof, and hybridization of the biotinylated cRNA on human HG-U133A arrays took place in accordance with the “Affymetrix User Guide” using Superscript II (from Gibco) and the High Yield cRNA Labeling Kit (from Enzo). The HG-U133A array in principle permits simultaneous mRNA analysis of about 22 600 human genes. The arrays were analyzed using the software MAS 5.0 (from Affymetrix) and Gene Spring 5.0 (from Silicon Genetics). FIG. 1 summarizes the genes expressed differentially between atrium and ventricle in all 6 investigated patients. The ratio of the normalized expression from atrium and ventricle is indicated, in each case as average for all 6 subjects.

The differential expression between atrium and ventricle found by means of array for the potassium channels TWIK-1, TASK-1, GIRK1, SK2 and PCN1 is verified by quantifying the mRNA in a real-time polymerase chain reaction (10). For this, the total RNA is isolated as described above from the human myocardium samples and in each case 1 μg thereof is converted with 1 unit of DNase I (from Gibco) at room temperature for 15 min. to remove contamination by genomic DNA. Inactivation of the DNase I takes place by adding 1 μl of EDTA (25 mM) and subsequent heating at 65° C. (10 min). The cDNA synthesis is then carried out in the same reaction mixture in accordance with the instructions for “SUPERSCRIPT-II RT cDNA synthesis kit” (from Gibco), and the reaction volume is made up to 200 μl with distilled water.

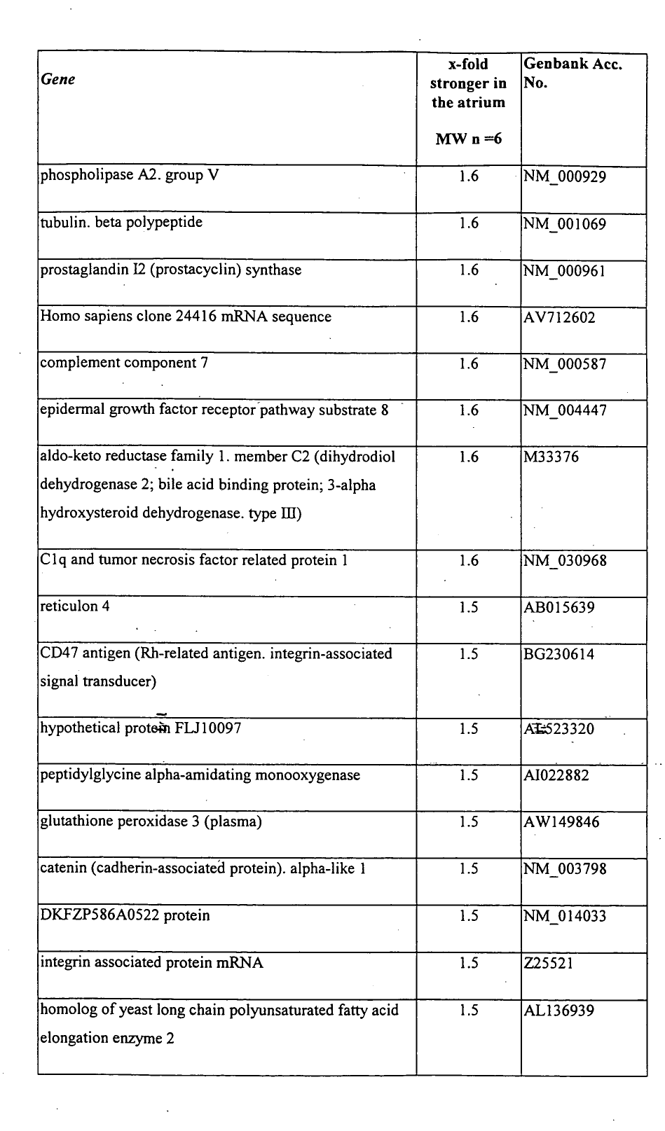

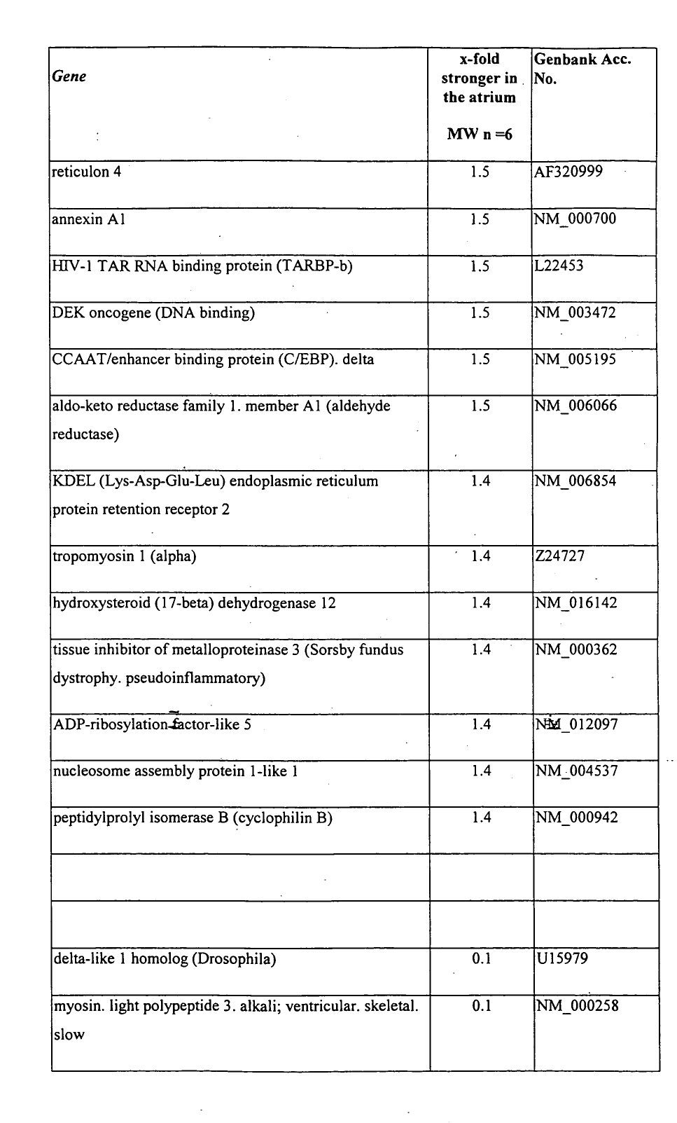

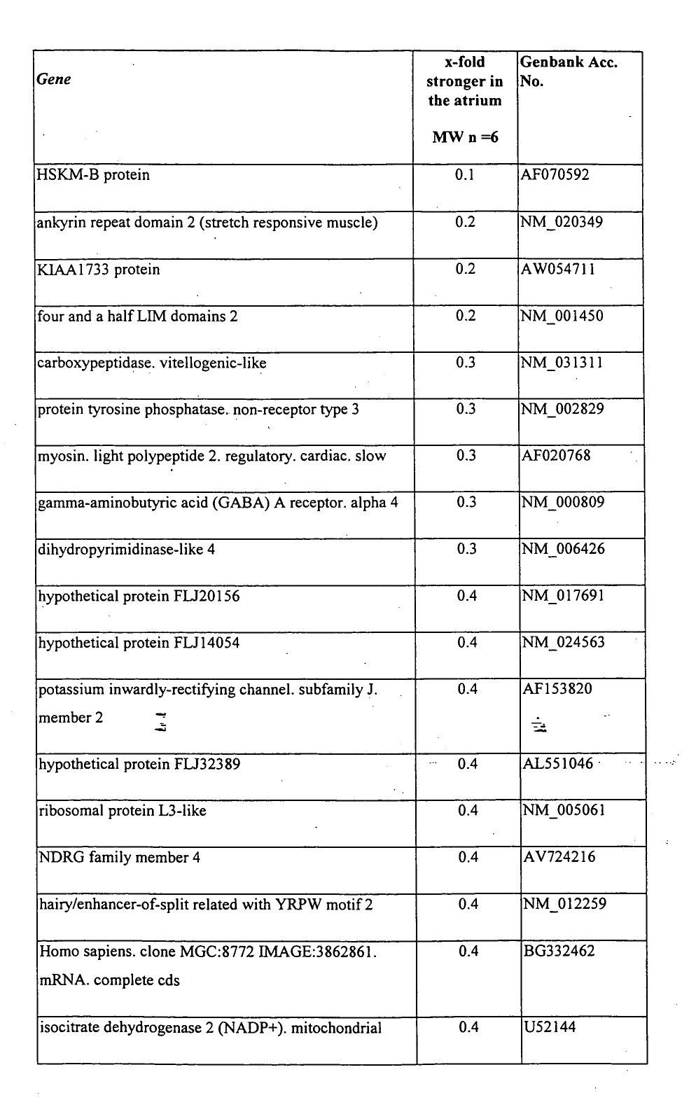

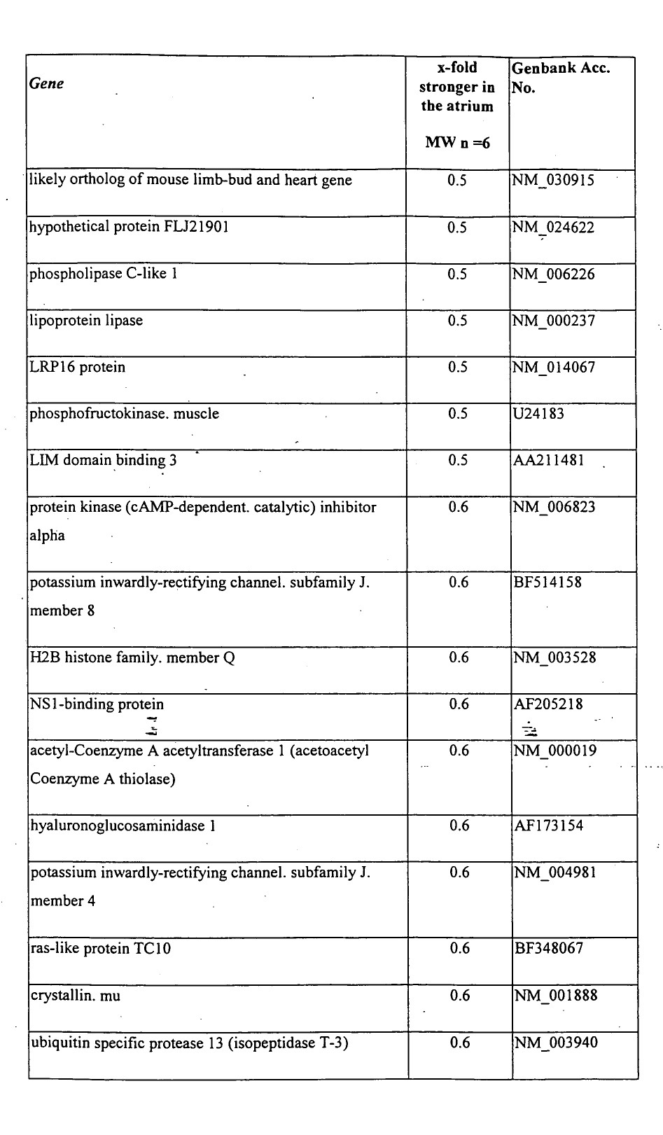

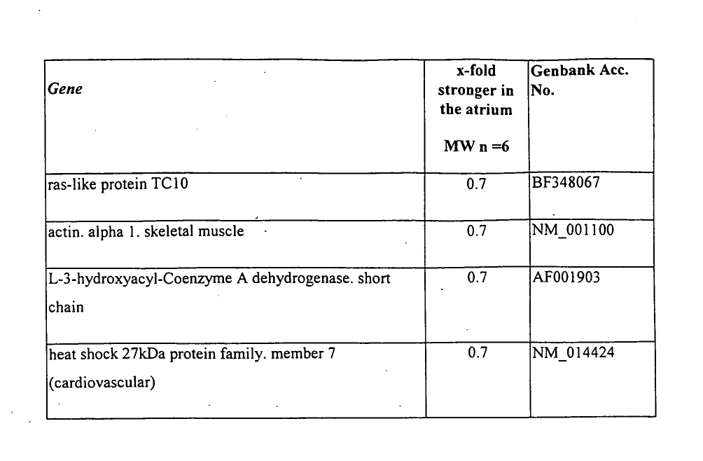

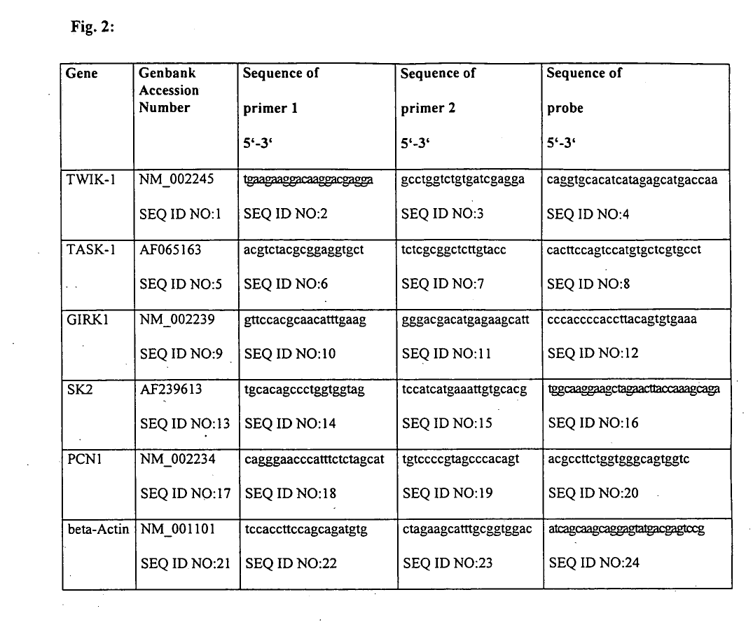

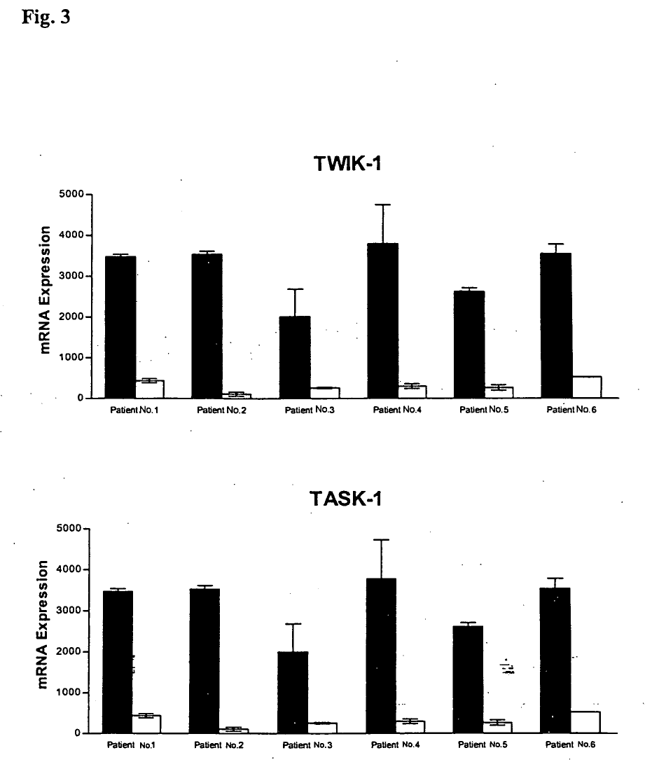

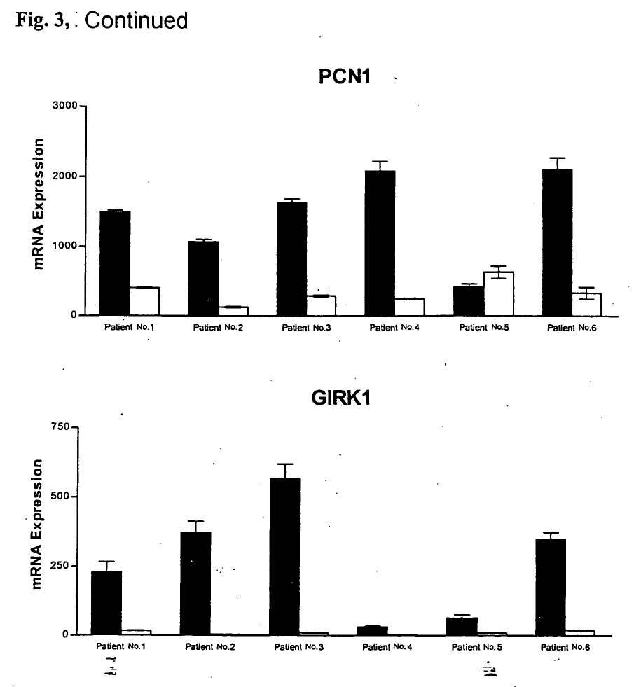

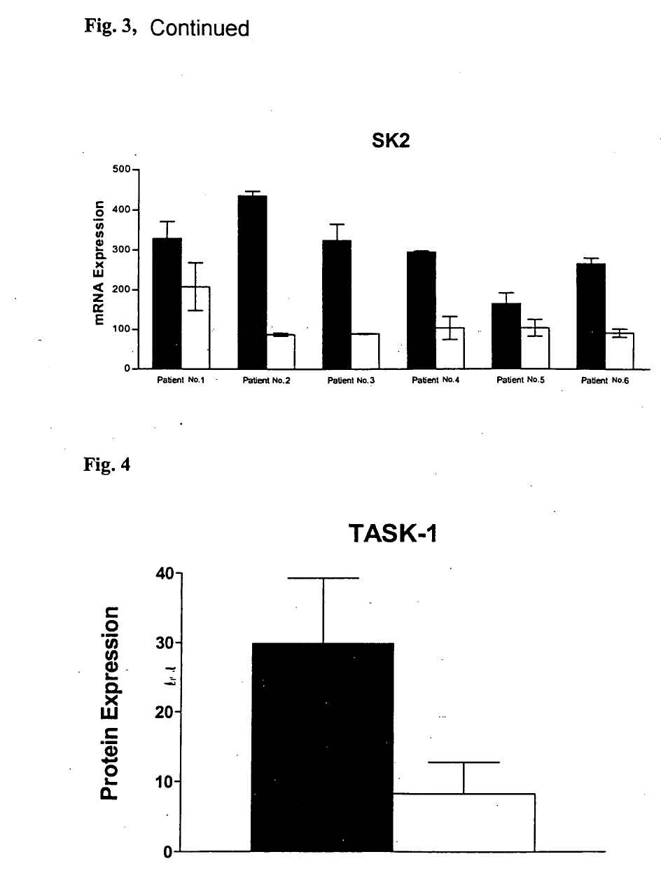

For the PCR, 7.5 μl of mixture of primer and probe and 12.5 μl of TaqMan reaction solution [Universal Master Mix (from Applied Biosytems] is added to 5 μl portions of the diluted cDNA solution. The final concentration of the primers is 300 nM in each case, and that of the probe is 150 nM. The sequences of the primers, and the Genbank accession numbers of the analyzed genes are indicated in FIG. 2. Suitable primer and probe sequences were identified using the program Primer Express 5.0 (from Applied Biosystems), and the PCR took place on an ABI Prism SDS 7700 instrument (from Applied Biosystems) in accordance with the manufacturer's instructions. The real-time PCR involves recording of the so-called Ct value which is obtained for the relevant gene in the investigated tissue. This corresponds to the cycle in which the fluorescence intensity of the released probe is about 10 standard deviations above the background signal. A lower Ct value means an earlier start of the amplification, i.e. more mRNA present in the original sample. To compensate for possible variations on the cDNA synthesis, the expression of a so-called “housekeeping gene” is also analyzed. Expression of the latter should be approximately the same in all tissues. The potassium channel expressions in atrium and ventricle were normalized uniformly by using β-actin. The dCt value is calculated for each gene and each tissue for graphical representation of the relative mRNA expression. The dCt value is the difference between the Ct value of the investigated potassium channel and the Ct value of the housekeeping gene in the respective tissue. The relative expression rE is calculated from this value by the following formula: rE=2^{(20-dCt)}. This is indicated in FIG. 3 as dimensionless number.

Protein expression was analyzed for the potassium channel TASK-1 using a commercially available antibody (from Santa Cruz). For this purpose, small pieces of tissue (about 50 mg) were homogenized in 1× PBS (with 1% Triton) and, after centrifugation and determination of the concentration (BCA-Tet, from Pierce), a Western blot was carried out (10% Nupage gel). Detection took place by means of the ECL system (from Amersham) using an HRP-conjugated anti-goat IgG antibody. The exposed film was evaluated by densitrometry in a bioimager (from Fuji). The result is indicated in FIG. 4 as dimensionless number.

### Example 2

### Identification of Potassium Channel Modulators

Potassium channel modulators are identified in a cellular assay in which CHO cells recombinantly express the respective ion channel and with use of the potential-sensitive Dye B from the “FLIPR membrane potential assay kit” (from Molecular Probes). Depolarization of the cells by a chemical substance leads to an increased uptake of Dye B and thus an increased intracellular fluorescence intensity. Hyperpolarization of the cell by a chemical substance by contrast leads to a decrease in the dye concentration in the cell and thus also a decrease in the fluorescence intensity, because the quantum yield of Dye B in aqueous solution is lower. Confluent cells are used for the measurement and, after removal of the medium, are loaded with Dye B at room temperature in accordance with the instructions of the kit manufacturer (Molecular Probes). The fluorescence is likewise measured at room temperature in a Fluobox (from Tecan) with an excitation wavelength of 520 nm and an absorption wavelength of 575 nm, as described for example in (11).

### Example 3

### Testing of the In Vivo Effect of Potassium Channel Modulators

The effect of the potassium channel modulators on the heart rate is investigated in anesthetized rats. For this purpose, male Wistar rats (250-300 g) are anesthetized with 10 mg/kg thiobutabarbitali i.p. (Inactin, Byk Gulden) and then sacrificed. After opening the thorax, the heart is exposed, and the right atrium is isolated and stored under a tension of 1 g in a Krebs-Henseleit solution (in a 10 ml organ bath) at 30° C. This solution is gased with Carbogen (95% O₂, 5% CO₂) at pH 7.2-7.4. The atria beat spontaneously and, after recording for a control period (parameter: rate), the test substances are administered in a dose series. The change in the rate compared with placebo-treated controls is evaluated for each dose.

### Example 4

### Potassium Channel Modulator Formulations

The potassium channel modulators can be converted in a known manner into conventional formulations such as tablets, coated tablets, pills, granules, aerosols, syrups, emulsions, suspensions and solutions, with use of inert, nontoxic, pharmaceutically acceptable carriers or solvents. In these cases, the therapeutically active compound should in each case be present in a concentration of 0.5 to 90% by weight of the complete mixture, i.e. in amounts which are sufficient to reach the stated dose range.

The formulations are produced for example by extending the active ingredients with solvents and/or carriers, where appropriate with use of emulsifiers and/or dispersants, it being possible for example when water is used as diluent where appropriate to use organic solvents as auxiliary solvents.

Administration takes place in a conventional way, preferably orally, transdermally, intravenously or parenterally, especially orally or intravenously. However, it can also take place by inhalation through the mouth or nose, for example with the aid of a spray, or topically via the skin.

It has generally proved advantageous to administer amounts of about 0.001 to 10 mg/kg, on oral administration preferably about 0.005 to 3 mg/kg, of body weight to achieve effective results.

It may nevertheless be necessary where appropriate to deviate from the stated amounts, in particular as a function of the body weight or of the mode of administration, of the individual behavior toward the medicament, the nature of its formulation and the time or interval over which administration takes place. Thus, it may be sufficient in some cases to make do with less than the aforementioned minimum amount, whereas, in other cases the stated upper limit must be exceeded. Where larger amounts are administered, it may be advisable to divide these into a plurality of single doses over the day.

### REFERENCES

1. Forth, Henschler, Rummel; Allgemeine und spezielle Pharmakologie und Toxikologie; Urban & Fischer Verlag Munchen, 8^{th} edition 2001, 429-433
2. Numaguchi H. et al., Probing the interaction between inactivation gating and Dd-solatol block of HERG, Circ. Res. 11 (2000) 1012-1018.
3. Nattel, S. et al., Evolution, machanisms, and classification of antiarrhythmic drugs: focus on class III actions, Am. J. Cardiol. 84 (1999) 11R-19R.
4. Workmann, A. J. et al., A K(ATP) channel opener inhibited myocardial reperfusion action potential shortening and arrhythmias.
5. Lesage, F. et al., TWIK-1, a ubiquitous human weakly inward rectifying K⁺ channel with a novel structure, EMBO J. 15 (1996) 1004-1011.
6. Duprat, F. et al., TASK, a human background K+ channel to sense external pH variations near physiological pH, EMBO J. 16 (1997) 5464-5471.
7. Stoffel, M. et al., Human G-protein-coupled inwardly rectifying potassium channel (GIRK1) gene (KCNJ3): localization to chromosome 2 and identification of a simple tandem repeat polymorphism, Genomics 21 (1994) 254-256.
8. Desai, R. et al., Ca²⁺-activated K⁺ channels in human leukemic Jurkat T cells. Molecular cloning, biochemical and functional characterization, J. Biol. Chem. 275 (2000) 39954-39963.
9. Tamkun M. et al., Molecular cloning and characterization of two voltage-gated K+ channel cDNAs from human ventricle, FASEB J. 5 (1991) 331-337.
10. Heid C. et al., Real time quantitative PCR, Genome Res. 6 (1996) 986-9954.
11. EP906572(B1)

1. A method for treating or preventing cardiac dysrhythmias (arrhythmias), coronary heart disease or hypertension, comprising administering to a patient a therapeutically effective amount of a modulator of the potassium channels TWIK-1, TASK-1, GIRK1, SK2 or PCN1.

2. A method for screening test compounds to identify modulators of the potassium channels TWIK-1, TASK-1, GIRK1, SK2 or PCN1 which are suitable for producing a medicament for the treatment and/or prophylaxis of cardiac dysrhythmias (arrhythmias), coronary heart disease or hypertension.

3. A pharmaceutical composition comprising a modulator or a plurality of modulators of the potassium channels TWIK-1, TASK-1, GIRK1, SK2 or PCN1 for the treatment and/or prophylaxis of cardiac dysrhythmias (arrhythmias), coronary heart disease or hypertension.

4. (canceled)

5. A method for treating cardiac dysrhythmias (arrhythmias), coronary heart disease, or hypertension, comprising administering to a patient a therapeutically effective amount of a modulator of gene products which are expressed in the human heart differentially between left atrium and left ventricle.
